(19) United States
(12) Patent Application Publication
(10) Pub. No.: US 2026/0317274 A1
(43) Pub. Date: Oct. 15, 2026

(54) UREA PRODUCTION PLANT WITH STRIPPER BYPASS

(71) Applicant: Stamicarbon B.V., Sittard (NL)

(72) Inventor: Rahul PATIL, Maastricht (NL)

(73) Assignee: Stamicarbon B.V., Sittard (NL)

(21) Appl. No.: 19/484,274

(22) PCT Filed: Apr. 11, 2025
(86) PCT No.: PCT/NL2025/050172

§ 371 Date: Nov. 13, 2025

(30) Foreign Application Priority Data
Apr. 11, 2024 (EP) 24169751.5
Dec. 19, 2024 (EP) 24221807.1

(51) Int. Cl. C07C 273/04

(52) U.S. Cl. CPC C07C 273/04

(57) ABSTRACT

A urea production plant and process with HP CO₂ stripping and MP urea solution passing through a tube bundle of the HP condensation zone, wherein a part of the urea solution from the reaction zone bypasses the HP CO₂ stripper.

### FIELD

The invention pertains to the production of urea.

### INTRODUCTION

The invention pertains to urea production according to the CO₂ stripping process. In urea plants of the stripping type, the high pressure (HP) urea synthesis section comprises a reaction zone, a stripper, and a condensation zone. Thermal stripping and CO₂ stripping are two types of stripping processes. In the CO₂ stripping process, a part or all of the CO₂ feed gaseous stream is used as strip gas in the HP stripper. A background reference for urea production processes in general is Ullmann's Encyclopedia of Industrial Chemistry, chapter Urea, 2010. As mentioned therein, the CO₂ stripping process has as advantage that the stripped urea solution has a relatively low N/C ratio, such that it can supplied directly to an LP (low pressure) dissociator of a low pressure recovery section. In particular, the gas from the LP dissociator can be condensed in an LP carbamate condenser in a carbamate solution against typically available cooling water without excess NH₃.

Plants according to the thermal stripping process, and plants without a HP stripper, require a medium pressure (MP) recovery section between the synthesis section and the low pressure recovery section.

An advantageous type of CO₂ stripping urea plant is the so-called Ultra Low Energy (ULE) plant. References for this type of plant include, e.g., the paper of Patil and Weipeng, “Experiences with LAUNCH® MELT™ Ultra-Low Energy plant operation”, Stamicarbon Symposium 18 May 2022, and US 2015/0119603.

In this ULE type of urea plant, the synthesis section comprises reaction zone, a CO₂ stripper, and a horizontal submerged condenser that is a shell-and-tube heat exchanger comprising a first and a second tube bundle and a shell-side space, with in operation condensation of HP gas from the stripper in the shell side space and with a medium pressure urea solution in the first tube bundle. The medium pressure urea solution is obtained by adiabatically flashing stripped urea solution from the HP stripper to medium pressure (with release of a flash gas stream) and comprises carbamate, and is heated in the first tube bundle, thereby decomposing at least part of the carbamate into NH₃ and CO₂. The stream from the first tube bundle is subjected to gas/liquid separation. The liquid (urea solution) is contacted with the gas from the flashing to decrease the N/C ratio of the urea solution, and supplied to the dissociator of the LP recovery section. The gas from the gas/liquid separation is condensed in an MP carbamate condenser in indirect heat exchange with a urea solution to be heated. Typically the MP carbamate condenser is a shell-and-tube heat exchanger; typically with the urea solution in the tube bundle and with the MP gas to be condensed in the shell. Said MP carbamate condenser also receives the gas from the gas/liquid contacting unit and off-gas from the reaction zone. Excellent energy efficiency is obtained since the heat of the steam in the shell side of the HP stripper is used three times: for stripping, for carbamate dissociation in the MP urea solution in the tube bundle of the HP carbamate condensation section, and for heating the urea solution in the tube bundle of the MP carbamate condenser (without intermediate use of steam as heat transfer fluid). Said paper Patil 2022 mentions that the design is optimized from CAPEX and OPEX point of view. Furthermore, the shell side steam temperature in the HP CO₂ stripper is lower than in the earlier designs, contributing to the low consumption of high pressure steam (equivalent 23 bar steam). Note that in practice, the steam pressure of the stripper is lower than in older designs, namely e.g. about 15 bar. The second tube bundle is used for raising LP steam, e.g. 3-5 bar steam.

However, there is a desire to modify existing urea plants of this type to increase their capacity, with a so-called revamp. For a revamp in generally, it is desired to make maximum use of the existing equipment of the plant and to achieve process improvements (capacity increase and/or improved energy efficiency) with minimum capital expenditure. There is also a desire for large capacity single-train urea plants where the HP equipment, e.g. HP stripper and/or pool reactor, are not too large for manufacture and transport. There is also a desire for urea plants with relatively small equipment of the HP synthesis section compared to the urea production capacity, also for newly built plants. For instance, sourcing considerations may differ between equipment for the HP section and equipment for the MP and LP section, as different types of steels can be used; in particular for HP equipment, more corrosion-resistant steel grades are necessary and higher pressure designs.

Furthermore, although the ULE type plant has been discussed herein with reference to the example design given in Patil 2022, variations are possible in the context of the present invention. For instance, the synthesis section may consist of a stripper and a pool reactor modified with two tube bundles (a pool reactor is a single horizontal vessel combining the reaction zone and condensation zone), but a separate horizontal submerged condenser and a vertical urea reactor may also be used. Further variations are possible.

Further references for the ULE type urea plant include WO 2023/158314 (mentioning an advantage of low biuret content of the formed urea, and describing use of the steam from the second tube bundle for heating the urea solution) and WO 2023/219506 (describing a configuring of the two tube bundles). In both references, the figures illustrated the first tube bundle as receiving flashed urea solution from the HP CO₂ stripper.

Mechanical aspects of the pool reactor preferably used for the ULE plant are described in the paper of M. Wagemans, “Mechanical aspects of the pool reactor for the Ultra-Low Energy plant”, Stamicarbon Symposium 18 May 2022. In particular, since the first tube bundle is exposed to corrosive, carbamate-containing liquid, at high temperatures, a specific design with a “distribution box” inside the vessel and with connecting ducts to the vessel shell can be used, to prevent corrosion that could occur if a carbamate-containing liquid is passed through a carbon steel tube sheet. A further reference for this design of the carbamate condenser is US 2020/0306663. Another mechanical design of the tube to tube-sheet joint is described in US 2015/0086440, which describes a configuration with a sleeve that is inserted in bore holes through the tube sheet.

### SUMMARY

The invention provides, in a first aspect, in an (first) embodiment a urea production plant comprising: a high pressure (HP) synthesis section comprising a reaction zone, a condensation zone, and a HP CO₂ stripper, wherein the condensation zone is provided by a shell-and-tube heat exchanger comprising a shell side space and a first tube bundle; a gas flow line for gas from the HP CO₂ stripper to said shell side space; a first liquid flow line for a first part of the urea solution from the reaction zone to said HP CO₂ stripper; a second liquid flow line for a second part of the urea solution from the reaction zone to an MP treatment section; wherein the MP treatment section comprises, in series for urea solution, an MP flashing section, the first tube bundle, an MP gas/liquid separation unit, and an MP gas/liquid contacting unit, and wherein said second liquid flow line bypasses said HP CO₂ stripper.

The invention also provides, in this first aspect, a urea production process carried out in a plant according to the first aspect of the invention, the process comprising: reacting NH₃ and CO₂ under urea-forming conditions in said reaction zone and condensation zone, and supplying all or a part, preferably a part, of the CO₂ feed to the HP CO₂ stripper.

The invention also pertains, in the first aspect, to a method of modifying an existing urea plant, the existing urea plant comprising: a high pressure (HP) synthesis section comprising a reaction zone, a condensation zone, and a HP CO₂ stripper, wherein the condensation zone is provided by a shell-and-tube heat exchanger comprising a shell side space and a first tube bundle; a gas flow line for gas from the HP CO₂ stripper to said shell side space, a first liquid flow line for urea solution from the reaction zone to said HP CO₂ stripper; a further liquid flow line for urea solution from the HP CO₂ stripper to an MP treatment section, wherein the MP treatment section comprises, in series for urea solution, an MP flashing section, the first tube bundle, an MP gas/liquid separation unit, and an MP gas/liquid contacting unit; wherein the method comprises: providing a second liquid flow line for a second part of the urea solution from the reaction zone to the MP treatment section, wherein said second liquid flow line bypasses said HP CO₂ stripper; preferably wherein the modified plant is a plant according to the first aspect of the invention.

The invention provides, in this first aspect, a urea production plant and process with HP CO₂ stripping and MP urea solution passing through a tube bundle of the HP condensation zone, wherein a part of the urea solution from the reaction zone bypasses the HP CO₂ stripper.

Also provided is, in a second aspect, a urea production plant wherein the MP treatment section (as specified above) is connected to receive urea solution from the HP synthesis section, preferably from the reaction zone and/or the HP CO₂ stripper; and provided is a urea production process carried out in that plant. Hence, in the embodiments of the second aspect, the second liquid flow line (used in the first embodiment as mentioned above) is optional. In an example configuration of this embodiment of the second aspect, the MP treatment section is connected to the HP stripper and the HP stripper receives all urea solution from the reaction zone.

Hence, in this second aspect, a urea production plant is provided, the plant comprising: a high pressure (HP) synthesis section comprising a reaction zone, a condensation zone, and a HP CO₂ stripper, wherein the condensation zone is provided by (is comprised in) a shell-and-tube heat exchanger comprising a shell side space and a first tube bundle. The plant further comprises, in the second aspect, a gas flow line for gas from the HP CO₂ stripper to said shell side space; a (first) liquid flow line for a first part of the urea solution from the reaction zone to said HP CO₂ stripper; and an MP treatment section. The MP treatment section is connected to receive urea solution from the HP synthesis section, preferably from the reaction zone and/or from the HP CO₂ stripper.

The MP treatment section comprises, in this second aspect, in series for urea solution, a medium pressure (MP) flashing section, the first tube bundle (the tube bundle comprised in said shell-and-tube heat exchanger), an MP gas/liquid separation unit, and an MP gas/liquid contacting unit. The MP treatment section further comprises an MP carbamate condensation section connected to receive gas from the MP gas/liquid separation unit and from the MP gas/liquid contacting unit. The MP condensation section comprises: a first MP carbamate condenser and a second MP carbamate condenser. The second MP carbamate condenser is connected to receive non-condensed gas from the first MP carbamate condenser. The MP treatment section comprises a connection (liquid flow connection) for MP carbamate (liquid) from the second carbamate condenser to the first MP carbamate condenser. The second MP carbamate condenser is in indirect heat exchanging contact with a first evaporation stage (evaporator) that receives urea solution. The first evaporation stage has an outlet for heated urea solution connected to a second evaporation stage that is in indirect heat exchanging contact with the first MP carbamate condenser. The plant comprises the first and second evaporation stage. This configuration of the MP treatment section is used in the second aspect and is preferably used in the first aspect of the invention.

The plant comprises, in the second aspect: a gas flow line for off-gas from the synthesis section, preferably from the condensation zone, to the second carbamate condenser, and a gas flow line for gas from the gas/liquid separation unit directly or indirectly to the first MP carbamate condenser, and/or a gas flow line for gas from the gas/liquid contacting unit directly or indirectly to the first MP carbamate condenser. These gas flow lines to the first MP carbamate condenser are provided, preferably, by a gas flow line from the gas/liquid separation unit to the gas/liquid contacting unit and a gas flow line from the gas outlet of the gas/liquid contacting unit to the first MP carbamate condenser. The plant of the first aspect preferably has these flow connections.

Preferences and details of the urea plant of the first aspect apply also to this second aspect, except that the second liquid flow line is optional in this second aspect. The preferences and details for the MP treatment section, in particular the MP carbamate condensation section of this second aspect,, also apply as preferences to the MP treatment section and MP carbamate condensation section of the urea plant of the first aspect.

### BRIEF DESCRIPTION OF THE DRAWINGS

FIG. 1 schematically illustrates an example urea production plant, process and method according to the first aspect of the invention.

FIG. 2 schematically illustrates an example urea production plant, process, and method according to the first aspect of the invention.

FIG. 3 schematically illustrates an example MP carbamate condensation section that can be used in the inventive plant, process, and method of the first aspect and that is used on the second aspect.

Any embodiments illustrated in the figures are examples only and do not limit the invention.

### DETAILED DESCRIPTION

The invention provides, in a first aspect, a method of modifying an existing urea plant (a so-called revamp). Briefly, an existing ULE type plant is modified by adding a bypass flow line for a part of the urea solution from the reaction zone that is supplied to the first tube bundle of the HP carbamate condenser, bypassing the HP CO₂ stripper, through the existing MP adiabatic flash vessel or through a newly added MP adiabatic flash vessel. Hence, in the modified plant, a first part of the urea solution from the reaction zone is supplied to the HP CO₂ stripper and a second part of the urea solution is supplied through the bypass flow line (i.e. the second liquid flow line) to the first tube bundle. The stripped urea solution from the HP CO₂ stripper can be processed in various ways: through a dedicated recovery section (by adding a recovery section), or can join the first part of the urea solution at the MP adiabatic flash unit, at the first tube bundle, or downstream of the first tube bundle. A capacity increase of up to 60% (at least) is feasible with limited additional equipment, usually the capacity increase is at least 10% (of the urea production capacity); capacity increases are relative to the design urea production capacity of the existing plant.

The N/C ratio of the urea solution at the reaction zone (or reactor) outlet is e.g. 2.8-3.8, e.g. 2.9-3.2, the N/C ratio is typically the same at the liquid inlet of the HP CO₂ stripper. The N/C ratio of the stripped urea solution is for example in the range 2.0 to 2.6. Therefore, compared to a reference ULE process wherein the first tube bundle receives only stripped urea solution (e.g. with N/C ratio of about 2.4), the N/C ratio of the MP urea solution in the first tube bundle is higher. This was found to very elegantly provide for better heat exchange from the condensing gas. Hence, a smaller heat exchanging surface can be used, and accordingly a relatively small HP carbamate condenser relative to urea production. In other words, very elegantly the plant capacity can be increased while using the existing ULE type HP carbamate condenser, in a revamp of a ULE type urea plant.

A background reference in this respect is US 2004/0116743A1. This document describes a method for increasing the capacity of existing urea processes by feeding a part of the urea synthesis solution to an additionally installed medium-pressure treatment zone comprising a dissociation zone, an adiabatic medium-pressure stripping zone (where the liquid is contacted with gas to rectify the N/C ratio, using a part of the CO₂ feed as gas), and a medium-pressure condensation zone. The revamping method of the present invention however concerns the revamping of an existing urea plant of the ULE type, i.e. an existing urea plant that already has a medium pressure dissociation zone (namely, the first tube bundle), a medium pressure gas/liquid contacting zone, and a medium pressure condensation zone. Indeed Patil 2022 refers to the ULE plant as having a “fully optimized” design.

The existing, modified, and inventive plant operate typically with a synthesis pressure in the range of 140-150 bar in the synthesis section, with each of the reaction zone, condensation zone and stripper operating in this range (shell side for condensation zone, tube side for stripper).

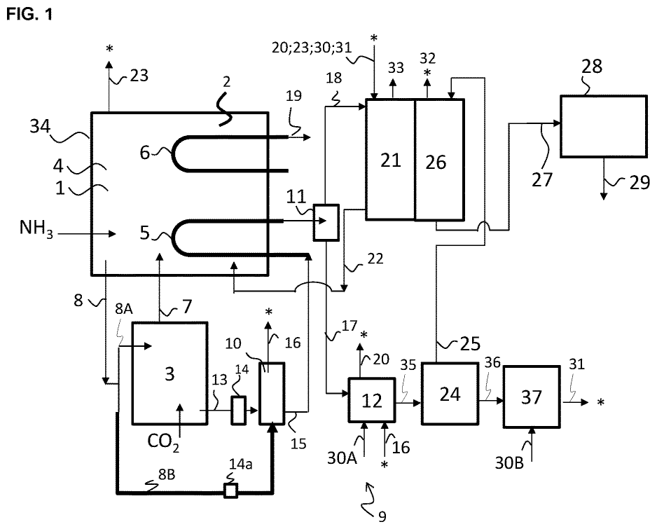

With reference to FIG. 1, the existing urea plant comprises a high pressure (HP) synthesis section comprising a reaction zone (1), a condensation zone (2), and a HP CO₂ stripper (3), wherein the condensation zone is provided by a shell-and-tube heat exchanger (34) comprising a shell side space (4) and a first tube bundle (5). The plant also comprises a gas flow line (7) for gas from HP CO₂ stripper (3) to said shell side space (4). The existing plant comprises a first liquid flow line (8A) for the urea solution (8) from the reaction zone to said HP CO₂ stripper. The existing plant also comprises a liquid flow line for stripped urea solution (13) from the HP CO₂ stripper to an MP treatment section (9).

The MP treatment section (9) comprises, in series for urea solution, an MP flashing section (10), the first tube bundle (5), an MP gas/liquid separation unit (11), and an MP gas/liquid contacting unit (12). One or more control valves can be present between the units. It is noted that the gas/liquid separation unit (11) and an MP gas/liquid contacting unit (12) can also be provided in a single combined unit, e.g. by a rectification column with a flash zone on top and a packed bed below the flash zone.

In the existing plant, all urea solution received by the first tube bundle originates from the HP CO₂ stripper, after suitable expansion of the stripped urea solution (13) to medium pressure using a valve (14). The MP adiabatic flashing section (10) in the existing plant contains (and usually consists of) an MP adiabatic flash vessel that has a liquid inlet for MP urea solution (in practice, a gas/liquid mixture from the valve (14)), a liquid outlet (15) and a separate gas outlet (16). The adiabatic flashing of the urea solution results in cooling, also considering the endothermic decomposition of carbamate into NH₃ and CO₂ upon pressure release. This cooling of the urea solution contributes to good heat exchange in the first tube bundle. In the existing plant, the gas stream (16) obtained by flashing stripped urea solution is rich in CO₂ (has a low N/C ratio) and the urea solution (15) accordingly has a relatively high N/C ratio. The flashed urea solution (15) is supplied, at MP, to the first tube bundle and comprises carbamate. It is noted that in known plants, the MP adiabatic flashing section typically consists of a single MP flash vessel and is accordingly referred to as a flash vessel, not as a flashing section.

The MP urea solution (15) is heated in the first tube bundle by heat exchange against the hot liquid phase in the shell-side space (where carbamate condensation occurs in an exothermic reaction). At least a part of the carbamate in the MP urea solution decomposes in the tube bundle by the heating. The gas/liquid mixture from the tube bundle is subjected to gas/liquid separation in a gas/liquid separation unit (11) which has a liquid outlet (17) for urea solution and a separate gas outlet (18).

In the existing plant, the urea solution (17) has a relatively high N/C ratio and is contacted with the gas stream (16) from the flashing, adiabatically, in the gas/liquid contacting unit (12), preferably with counter-current contact. Thereby, the N/C ratio of the urea solution decreases. The gas/liquid contacting unit (12) has a liquid outlet (35) for urea solution and separate a gas outlet (20). The urea solution (35) is supplied to a dissociator (24) of the LP recovery section. The gas (36) from the LP dissociator is condensed in an LP carbamate condenser (37) to form an LP carbamate solution (31). The urea solution (25) from the LP dissociator is typically supplied to an evaporation stage for water removal, usually after expansion to atmospheric pressure and further pressure reduction to a pressure of less than 1.0 bara.

The relatively low N/C ratio of the urea solution (35) is advantageous for the condensation in the LP carbamate condenser.

The MP treatment section (9) further comprises an MP carbamate condensation section (21) that is connected to receive the flash gas stream (18) and the gas (20) from the gas/liquid contacting unit, and the non-condensed gas (23) from the synthesis section (from the reaction zone and/or condensation zone) and the LP carbamate solution (31). The MP carbamate condensation section (21) has a liquid outlet (22) for MP carbamate solution that is supplied, directly or indirectly, to the shell side space (4) of the HP carbamate condenser. The MP carbamate condensation section (21) may comprise one or more carbamate condensers, e.g. two or more carbamate condensers in series or in parallel. The MP carbamate condensation section (21) also has an outlet for non-condensed gas (33); for example this gas is supplied to an absorber.

Preferably, at least one of the carbamate condensers is in heat exchanging contact with an evaporator (26) that is used for heating the urea solution (25) obtained directly or indirectly from the LP recovery section, for removal of water vapor (32) in order to increase the urea concentration. For instance, the plant comprises a shell-and-tube heat exchanger with the urea solution (25) to be heated in the tubes, at a pressure below 1.0 bara, and with the gas (18) to be condensed at medium pressure in the shell. In the evaporator, water is removed from the urea solution by the heating. The heated urea solution (27) from the evaporator (26) is e.g. a urea melt with e.g. at least 90 wt. % urea (including biuret), and is supplied, optionally after further evaporation in an evaporator, for instance to a finishing section (28) where it is solidified into a solid urea product (29). The finishing section is e.g. a granulator or prilling tower. The melt (27) can also be used, in part or entirely, for producing melamine, e.g. when having at least 98 wt. % urea (including biuret).

The modification of the existing plant (the revamp) involves adding a second liquid flow line (8B) for a second part of the urea solution from the reaction zone to the MP treatment section (9), which second liquid flow line bypasses the HP stripper (3). The modification may involve adding separate liquid outlet of the reaction zone for said second part of the urea solution and/or adding a liquid divider for dividing the urea solution (8) from the reaction zone in a first and second part. The second liquid flow line (8B) preferably has a dedicated valve (14A) for expansion of the urea solution from HP to MP.

The invention also pertains to the modified plant, and also generally to the inventive plant with the MP treatment section and the first and second liquid flow line for urea solution from the reaction zone, the second liquid flow line bypassing the HP CO₂ stripper and connected to the MP treatment section, wherein the MP treatment section comprises the first tube bundle of the HP carbamate condenser. The inventive plant can therefore also be a newly built plant. For newly built plants, an advantage is that the HP condensation zone and the HP stripper are relatively small compared to urea production capacity.

Preferably, the HP carbamate condenser also comprises a second tube bundle that is used for raising steam, in particular low pressure (LP) steam.

In an example embodiment, the synthesis section comprises a pool reactor as said shell-and-tube heat exchanger (34), wherein the pool rector is provided by a horizontal vessel with vertical partition plates to divide the vessel in compartments.

In a further embodiment, the condensation zone (2) is provided by a so-called pool condenser, i.e. a shell-and-tube heat exchanger (34) with, in the ULE plant, two U-shaped tube bundles, and typically with a liquid outlet at the top.

Highly preferably, the non-stripped second part of the urea solution is subjected to MP adiabatic flashing upstream of the first tube bundle, with gas/liquid separation, and with selectively the liquid supplied to the first tube bundle. Removal of the gas resulting from the expansion of the urea solution comprising carbamate from HP to MP upstream of the first tube bundle contributes to efficient heating in the first tube bundle.

The gas from the adiabatic flash step is supplied to the MP carbamate condensation section (21). Preferably, the urea solution from the HP CO₂ stripper is also subjected to MP adiabatic flashing, with gas/liquid separation, and with the resulting gas also supplied to the MP carbamate condensation section (21), more preferably to the same MP carbamate condenser for combined condensation.

Thereby advantageously an improved N/C ratio is achieved for the combined condensation, considering that the stripped urea solution (13) and the non-stripped second part of the urea solution (8B) have different N/C ratios. More preferably, the combined condensation is carried out in indirect heat exchange (through a wall) with urea solution to be heated. The optimum N/C ratio contributes to effective heating of the urea solution. Optionally, the flashing of the non-stripped urea solution and stripped urea solution is done in the same MP flash unit; or in different MP flash units.

The urea solution of the second flow line (8B) is preferably subjected to MP flashing and is hence supplied to the MP flash section (10), which may comprise one flash vessels or two or more flashing vessels in parallel. In case of a single MP flash vessel, this flash vessel receives both non-stripped and stripped urea solution, and a part or all of the urea solution from the flash vessel is supplied to the first tube bundle. In an embodiment, a first part of the urea solution from the single flash vessel is supplied to the first tube bundle and a second part of the flashed urea solution bypasses the first tube bundle and is supplied to, e.g., the gas/liquid contacting section, or to the LP recovery section.

In case of a first and a second MP flash vessel arranged parallel, for example the first vessel receives a stripped urea solution and the second vessel receives non-stripped urea solution, or the first vessel receives combination of stripped and non-stripped urea solution and the second receives either stripped or non-stripped urea solution, or both receive a combination of stripped and non-stripped urea solution, e.g. in different or the same ratios.

The inventive plant comprises a liquid flow line from the second liquid flow line, through the flash section, to the first tube bundle; hence the first tube bundle receives at least a part of the flashed MP urea solution originating from the second liquid flow line.

Generally, for flash sections with one flashing unit or two or more parallel flashing units, a part or all of the flashed MP urea solution is supplied to the first tube bundle. Optionally, a part of the flashed MP urea solution bypasses the first tube bundle and is supplied to the gas/liquid separator or the gas/liquid contacting unit of the MP treatment section, or possibly even directly to a dissociator in the LP recovery section or to a dissociator of an additional LP recovery section.

In the event of two MP flash units in parallel operating with different N/C ratio, preferably flashed urea solution from the flash vessel with the higher N/C ratio is supplied to the first tube bundle and preferably any flashed urea solution bypassing the first tube bundle originates from the flash vessel with lower N/C ratio. Generally, the stripped urea solution has a lower N/C ratio than the non-stripped urea solution.

As a general preference, all MP flash steps are adiabatic.

A part or all of the gas from the flash section is supplied to the gas/liquid contacting unit. In the mentioned case of parallel flash units with different N/C ratio, for instance flash gas from the flash unit with the lower N/C ratio is supplied to the gas/liquid contacting unit and flash gas from the other flash unit is supplied directly to the MP carbamate condensation section, preferably to an MP carbamate condenser also receiving gas from the gas/liquid contacting unit.

The invention also pertains to the urea production process carried out in said inventive plant, wherein the plant has the MP carbamate condensation section and/or an LP carbamate condensation section. The LP carbamate condensation section receives gas from one or more LP dissociator. The LP dissociators receive urea solution from the synthesis section, preferably the one or more LP dissociators in total receive urea solution originating from the first and the second liquid flow line. The MP carbamate condensation section receives gas from one or more other units of the MP treatment section.

The process involves reacting NH₃ and CO₂ to form urea, i.e. under urea-forming conditions, in said reaction zone (1) and condensation zone (2). In the first aspect, the process comprises supplying a first part of the urea solution from the reaction zone to the HP CO₂ stripper through the first liquid flow line, and letting a second part of the urea solution from the reaction zone bypass the HP CO₂ stripper through the second liquid flow line to the MP treatment section.

In the second aspect of the invention, the process comprises supplying a part or all of the urea solution from the reaction zone to the HP CO₂ stripper through the first liquid flow line, and preferably letting a second part of the urea solution from the reaction zone bypass the HP CO₂ stripper through the second liquid flow line to the MP treatment section.

Preferably, at least 10 wt. % of the urea solution from the reaction zone, on the basis of urea, is supplied to the second liquid flow line.

In the first aspect of the invention, the process preferably supplying a part of the CO₂ feed to the HP CO₂ stripper (3), and supplying a part of the CO₂ feed, as gas to the LP carbamate condensation section and/or to the MP carbamate condensation section, e.g. with at least 10 mol. % of the CO₂ feed to the LP and MP carbamate condensation section in total.

In a preferred embodiment, X wt. % of the urea solution from the reaction zone, on the basis of urea, is supplied to the first liquid flow line and to the HP CO₂ stripper and the amount of CO₂ supplied to the HP CO₂ stripper is Y % of the CO₂ feed of the plant, wherein Y=0.8<X<1.2, more preferably Y=0.9<X<1.1. Preferably, X is max. 90%., i.e. preferably at least 10% of the urea solution is supplied to the second liquid flow line and bypasses the HP CO₂ stripper. For example, if 70% of the urea solution is supplied to the first liquid flow line (30% bypasses the HP CO₂ stripper), preferably 56 to 84% of the CO₂ feed of the plant is supplied to the HP CO₂ stripper.

In the first aspect of the invention, the amount of CO₂ supplied to the HP CO₂ stripper is preferably 0.9-1.1 mol CO₂ per mol urea supplied to the HP CO₂ stripper, preferably about 1.0 mol. This preference also applies to the second aspect. This molar ratio is same as in a standard CO₂ stripping type urea plant wherein all urea solution from the reactor is subjected to HP stripping in the HP CO₂ stripper using all of the CO₂ feed as strip gas.

The process of the first aspect comprises supplying urea solution from the the reaction zone to the MP flashing section, and also optionally stripped urea solution from the HP CO₂ stripper to the MP flashing section.

The process of the second aspect generally comprises supplying urea solution from the synthesis section, preferably from the reaction zone and/or from the HP stripper, to the MP flashing section.

The process generally comprises supplying urea solution from the MP flashing section to the first tube bundle, from the first tube bundle to the MP gas/liquid separation unit, and from the MP gas/liquid separation unit to the MP gas/liquid contacting unit. The urea synthesis conditions are typically a pressure of at least 100 bar or at least 120 bar, typically 130-150 bar; and typically at a temperature of at least 160° C., or at least 170° C., or e.g. 180-190° C. (reactor outlet temperatures). The N/C ratio in the synthesis section is typically 2.9-3.5, preferably 3.0-3.2 (on the basis of the theoretical initial mixture).

Advantageously, the urea solution at the inlet of the first tube bundle has a free N/C ratio of at least 3.2, e.g. in the range 3.2 to 3.8; the free N/C ratio refers to the N/C ratio of the solution as molar ratio of NH₃ and CO₂, inclusive of NH₃ and CO₂ present as carbamate in the solution but excluding urea and biuret. Preferably, the urea solution at the inlet of the first tube bundle has a temperature in the range 125-140° C., or in the range 130-140° C., more preferably 130-135° C. This relatively low temperature is achieved by flashing the non-stripped urea solution, in particular to a medium pressure in the range 20 to 35 bara. The low temperature and high N/C ratio contribute to improved heat exchange in the HP carbamate condenser, permitting the processing of more MP urea solution in the first tube bundle with a fixed heat exchanging area of the tube bundle. In a standard ULE process, the temperature of the flashed stripped urea solution is e.g. 145-150° C.

Preferably in this process of the first aspect, at least 10% or at least 15% of the urea solution from the reaction zone bypasses the HP CO₂ stripper and is supplied, through MP flashing, to the first tube bundle. Preferably, the urea solution received by the first tube bundle comprises at least 10%, or at least 20% or at least 50% or at least 60%, e.g. up to 100%, flashed urea solution originating from the second flow line, i.e. flashed non-stripped urea solution. This contributes to the advantageous high N/C ratio of the urea solution in the first tube bundle. Hence, preferably, the urea solution received by the first tube bundle is for at least a part, more preferably a large part, provided by the flashed urea solution originating from the second flow line. Preferably at least 50% or at least 60%, of the flashed urea solution originating from the second flow line is supplied to the first tube bundle. In an example embodiment, max. 60% of the urea solution from the reaction zone bypasses the HP CO₂ stripper, or max. 35% thereof, and is supplied, through the MP flashing, to the first tube bundle.

Furthermore, for example the synthesis section is operated with an N/C ratio of 3.05-3.15, thereby further contributing to the high N/C ratio of the urea solution in the first tube bundle.

In the inventive process, the HP CO₂ stripper is preferably operated with stripping efficiency of at least 70%, e.g. 70-80%, more preferably 75-80%. This is higher than in a standard ULE process (typical stripping efficiency: 60%-65% when operating at 100% design capacity). The higher stripping efficiency can be provided by using (saturated) steam with sufficiently high pressure in the shell side of the HP CO₂ stripper. Thereby, more carbamate is decomposed in the HP CO₂ stripper and condensed in the HP carbamate condenser, with synergistically efficient cooling by the MP urea solution in the first tube bundle (as well as by cooling fluid in the second tube bundle). The stripped urea solution has a free N/C ratio of e.g. 2.0-2.3.

These preferences apply, in particular, in embodiments with a single flashing unit in the flashing section (and e.g. with all flashed urea solution to the first tube bundle) and in embodiments with two or more flashing units in parallel. The revamped/inventive plant is operated for instance with 16-20 barg (e.g. about 18 barg) (saturated) steam in the shell side of the HP CO₂ stripper, compared to about 14 barg in the existing ULE plant. The revamped/inventive plant is operated for instance with a stripping efficiency of the HP CO₂ stripper of at least 70%, e.g. 70-80%, preferably 75-80%.

The revamped/inventive plant is operated e.g. with a partial pressure of the inerts in the reaction zone (or reactor) of 3-10% of the total pressure in the reaction zone, preferably 3-8%. Herein inerts refers to gases other than CO₂, NH₃ and H₂O. This relatively lower inerts partial pressure improves heat transfer to the tube bundles of the condensation zone. Very advantageously, the resulting higher amount of condensable gas in the synthesis off-gas is condensed in the MP condensation zone with process-process heat recovery.

In an embodiment, the modification of the existing urea plant involves no installation of an additional reactor. For example, the existing urea plant contains a single pool reactor and the modified plant also contains only the pool reactor as providing the reaction zone. Very advantageously, a capacity increase can still be obtained, e.g. at least 10% and e.g. up to about 35%, by increasing throughput (flow rate) in the synthesis section, in combination with letting a part of the urea solution bypass the stripper.

Hence, very advantageously a urea production capacity increase of up to approximately 35% can be achieved without addition of HP equipment in the synthesis section, and without modification of the shell-and-tube heat exchanger (34) used as condensation section and without modification of the HP CO₂ stripper.

In this embodiment of the revamp method, the HP reaction volume is not increased. Capacity increase is achieved in this embodiment exclusively by higher throughput in combination with increased recovery.

In a further embodiment, additional reaction volume is installed in the synthesis section as part of the revamp, i.e. an the plant comprises an additional reactor and the method of modifying the plant comprises installing this additional reactor. For instance, an additional vertical urea reactor is installed in the urea solution flow line form the pool reactor to the HP CO₂ stripper. In this manner, larger capacity increases can be achieved, of e.g. up to 60% increase.

Accordingly, in the modified and inventive plant, the reaction zone is in an embodiment provided by two or more reactors, which can be arranged in series or in parallel, and wherein for instance a combination a pool reactor and a vertical reactor is used (the pool reactor also comprising the condensation zone). An advantage of this configuration for a newly built plant is that the size of the pool reactor can be small relative to plant capacity. Moreover, process conditions can be optimized in each reactor.

In a preferred embodiment, the modified plant involves a vertical urea reactor connected to receive urea solution from a pool reactor. Preferably, the liquid inlet at the bottom of the vertical urea reactor is placed lower than the liquid outlet of the pool reactor, e.g. the vertical urea reactor is placed on ground level.

Optionally, a part of the HP CO₂ feed is supplied to the vertical urea reactor to provide heat for the endothermic urea formation reaction.

Preferably, in this embodiment, the plant comprises an MP ammonia condenser and an MP ammonia recycle line from the ammonia condenser to the reactor, which MP ammonia recycle line is separate from the MP carbamate recycle line. An advantage of separate ammonia recycle is the lower amount of water recycle, a low water recycle is advantageous for the urea yield in the synthesis section. The MP ammonia condenser receives non-condensed gas from the MP carbamate condensation section. The MP ammonia condenser is more preferably used in combination with supplying a part of the HP CO₂ feed is supplied to the vertical urea reactor. In this preferred embodiment, the CO₂ feed is in part supplied to the HP CO₂ stripper, usually in a molar ratio to urea in the stripper of 0.9-1.1), and in part to the MP carbamate condensation section and/or HP reactor. In a advantageous embodiment, at least 10% of the CO₂ feed, e.g. 10-25%, is supplied to the reactor, e.g. to the added reactor. Suppling a part of the CO₂ feed to the reactor may help avoid inferior operation (e.g. flooding) of the HP CO₂ stripper and can also be used to provide heat of reaction to the reactor, especially in embodiment wherein the reactor is placed on ground level.

Advantageously, the existing urea solution handling units of the MP treatment section are often no bottleneck for the increased urea solution flow up to 35% capacity increase. For example, both the stripped urea solution and the non-stripped urea solution from the second flow line are supplied to the same (existing) flash vessel of the MP flash section.

The capacity of the MP carbamate condensation section can be increased in customary ways, e.g. by adding an MP carbamate condenser. Capacity of the LP recovery section and of downstream sections can be increased as customary for revamp methods.

This embodiment is illustrated in FIG. 1, wherein the bypass flow line (8B) and the stripped urea solution (13) are both supplied to the same MP flash unit of the flashing section (10). Moreover, in FIG. 1, all flashed urea solution is supplied to the first tube bundle.

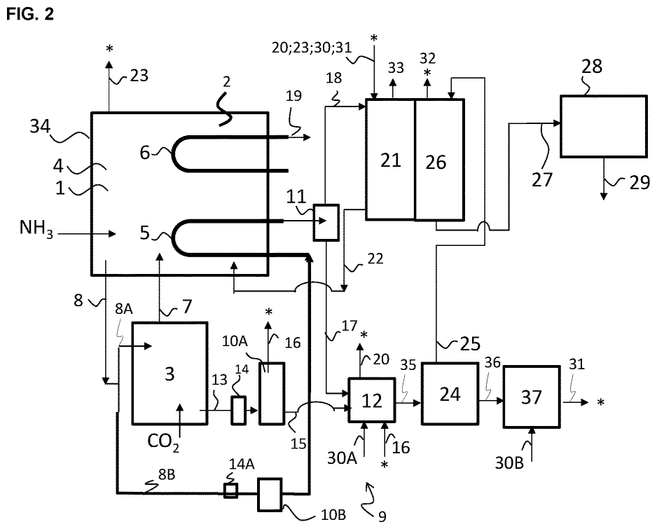

In a further embodiment of the inventive or modified plant, illustrated in FIG. 2, the second part of the urea solution is supplied, through the bypass second liquid flow line (8B) to a second MP flash vessel (10B) of the MP flash section (10) and the first MP tube bundle, at the inlet thereof, is connected exclusively to this second MP flash vessel (10B), that is arranged in parallel to a first MP flash vessel (10A). The stripped urea solution (13) is flashed in the first MP flash vessel (10A) and the flashed stripped urea solution (15) bypasses the first tube bundle and is supplied to the gas/liquid contacting unit (12). The second liquid flow line (8B) for example comprises a urea reactor, e.g. a vertical urea reactor, upstream of the valve (14A). Thereby the volume of the reaction zone can be increased. The revamp according to this embodiment would involve adding the second liquid flow line connected to the first tube bundle, adding an MP flash vessel, and connecting the HP stripper liquid outlet through an MP flash vessel (existing or added) to the existing gas/liquid contacting unit (12).

It is observed that the flashed urea solution from the parallel MP flash vessels can be combined and distributed in various ways. However, it is advantageous if the first tube bundle receives urea solution with higher N/C ratio. The first or the second MP flash vessel can be additionally installed during the modification of the existing urea plant. The installation of an MP flash vessel is in particular advantageous if an additional reactor is installed as part of the modification of the existing urea plant.

In a preferred embodiment, 10 to 40% of the urea solution from the reaction zone flows through the second liquid flow line, bypassing the HP stripper, and the stripper is operated with stripping efficiency of 70-75%, and stripped urea solution and urea solution from the second liquid flow line is flashed and supplied as combined stream to the first tube bundle, as in FIG. 1. Especially for stripping efficiency in this range, the combined urea solution contributes to good carbamate dissociation in the first tube bundle. The reaction zone is provided e.g. only one unit.

In a further preferred embodiment, 25 to 60% of the urea solution from the reaction zone flows through the second liquid flow line, bypassing the HP stripper, and the stripper is operated with stripping efficiency of 75-80%, and stripped urea bypasses the first tube bundle, and the first tube bundle only receives urea solution from the second liquid flow line, as in FIG. 2. Especially for high stripping efficiency operation of the stripper, letting the stripped urea solution bypass the first tube bundle contributes to good carbamate dissociation in the first tube bundle. The reaction zone is provided e.g. by at least two units in series.

In the embodiment of FIG. 2, the stripped urea solution does not pass through the first tube bundle, yet is not supplied directly to the LP recovery section as in a standard CO₂ type stripping plant either. Rather it is treated in a particular manner in this embodiment of the invention.

In a preferred embodiment, a part or all of the stripped urea solution bypasses the first tube bundle and is subjected to MP adiabatic flashing and subsequently MP gas/liquid contacting. Thereby advantageously, the combined urea solution supplied to the LP recovery section has a lower N/C ratio and the water recycle to the synthesis section is reduced.

It is observed that stream (15) can have a free N/C ratio of about 3.1, while the stream leaving the first tube bundle (5) can have a free N/C ratio of about 6.0. Contacting both liquids counter-currently with mainly fresh CO₂ enhances the free N/C ratio of the combined urea solution (18) to about 2.7, which is more suitable for processing in an LP recovery section including an LP condensation section.

Preferably the gas from this flashing is supplied to the MP carbamate condenser, thereby judiciously improving the N/C ratio for the condensation of the gas released in the first tube bundle.

Preferably the gas from the flashing is used for gas/liquid contacting of at least part of the total MP urea streams, preferably for said gas/liquid contacting of the stripped urea solution bypassing the first tube bundle.

In an embodiment wherein flashed stripped urea solution bypasses the first tube bundle, optionally the flashed stripped urea solution is supplied, directly or indirectly, to a first LP dissociator. Preferably in this embodiment, the urea solution from the first tube bundle, originating from the second liquid flow line bypassing the HP stripper, is supplied, directly or indirectly, to a second LP dissociator, without mixing of said urea solutions. Preferably in this embodiment, the flashed stripped urea solution is subjected to gas/liquid contacting and the urea solution from the first tube bundle is subjected to gas/liquid contacting in separate parallel units before being supplied to the respective LP dissociator. Thereby, the second LP dissociator will receive only urea solution originating from the second liquid flow line, i.e. without having passed through the HP stripper. The urea solution from the second LP dissociator then advantageously can have low metal content and low biuret content and is e.g. used in part or entirely for making Diesel Exhaust Fluid (DEF) or DEF precursor. Preferably, the urea solution from the second LP dissociator for making a (liquid or solid) urea product with less than 0.95 wt. % or less than 0.80 wt. % biuret relative to urea. A low biuret content and low metal content are important for meeting the specifications for DEF.

A standard for DEF is ISO 22241-1:2006 (version of 15 Oct. 2006). DEF should have low biuret content and also low metal content. DEF refers to an aqueous solution of urea for use in the selective catalytic reduction of NOx from exhaust gas (SCR solution). ISO 22241-1:2006 specifies biuret as max. 0.3 wt. % on the basis of 31.8 wt. % urea aqueous solution.

The urea solution from the first LP dissociator is for instance concentrated to a urea melt and this urea melt is, in a preferred embodiment, supplied in part or entirely, to a melamine production unit. The relatively higher biuret content of this urea solution form the first LP dissociator is suitably acceptable for melamine production. Preferably in this embodiment, the urea solution from the HP stripper is processed separately from the urea solution from the second liquid flow line. The biuret content of the urea solution from the first LP dissociator is for instance at least 0.02 wt. % (percentage point) or at least 0.05 wt. % (percentage point) higher than in the urea solution from the second LP dissociator, percentages based on anhydrous urea.

Also in case of a single LP dissociator, or in case of different configurations with multiple LP dissociators, a part of the urea solution can be used for making DEF. Making DEF may involve adding an aqueous stream to the urea solution to dilute the urea solution, e.g. to about 30-35 wt. % urea, which is the typical desired concentration for some types of DEF.

In case of multiple LP dissociators, preferably the gas from the dissociators is subjected to combined condensation to form a carbamate solution that is supplied to the MP carbamate condensation section.

As a general preference, at least a part of the CO₂ feed is supplied, directly or indirectly, as gas, to the MP carbamate condensation section (21) and/or directly or indirectly, as gas, to the LP carbamate condensation section (37). For example, said part is at least 10 mol. % of the CO₂ feed, or at least 20 mol. % thereof, for the MP and LP carbamate condensation section in total. Preferably, the LP carbamate condensation section receives gas from one or more LP dissociators that receive, in total, urea solution originating from the first and from the second flow line, e.g.

from the combined urea solution. Supplying gaseous CO₂ to the LP carbamate condensation section advantageously reduces the N/C ratio to achieve sufficient carbamate condensation without excess uncondensed NH₃. The MP carbamate condensation section preferably receives LP carbamate solution from said LP carbamate condensation section (which in turn preferably receives gas from LP dissociation in one or more LP dissociators of all the urea solution originating from the first and second flow line), i.e. a relatively large amount of LP carbamate solution, such that the N/C ratio in the MP carbamate condensation section (21) is relatively low.

In embodiments, a part (30) of the CO₂ feed is supplied, as MP gas stream, to the MP carbamate condensation section (21), e.g. to reduce the N/C ratio (so as to avoid the need for a separate NH₃ condenser) and/or to reduce the load on the HP stripper.

Note that in an optional embodiment, a separate NH₃ condenser can be used, i.e. for uncondensed MP gas (33), with a separate ammonia recycle to the synthesis section, in particular to the reactor. The ammonia recycle is advantageously substantially water-free. This can be useful especially in larger revamps (capacity increase of more than 30%). In this embodiment, preferably a part of the CO₂ feed is supplied to the reactor.

Preferably, the inventive or modified plant comprises a supply line (30) for an MP CO₂ feed gas stream directly or indirectly to the MP carbamate condensation section (21) and/or a supply line for a CO₂ feed gas stream to the LP carbamate condensation section (37), more preferably both. The inlet of the supply line is connected e.g. to an MP CO₂ compressor or to the HP CO₂ compressor of the synthesis section. The revamp preferably involves adding either or both supply lines, and suitably also adding the MP CO₂ compressor.

In an embodiment, at least a part (30A) of the MP CO₂ feed gas stream, or all thereof, is supplied through the gas/liquid contacting unit (12) to the MP carbamate condenser, and is hence contacted with the MP urea solution from the first tube bundle upstream (for gas) of the MP carbamate condensation section; preferably the contacting is adiabatically. Thereby, MP CO₂ feed gas is used advantageously for decreasing the N/C ratio of the MP urea solution from the first tube bundle.

Preferably, in an embodiment wherein a part of the urea solution from the flashing section bypasses the first tube bundle, e.g. wherein flashed stripped urea solution bypasses the first tube bundle, this urea solution is subjected to contact with a part or all of the MP CO₂ feed gas stream, e.g. in the same gas/liquid contacting unit or in a second gas/liquid contacting unit, to decrease the N/C ratio of the urea solution after the flashing.

Thereby, preferably stripped urea solution which does not pass through the first tube bundle is subjected to gas/liquid contacting with MP CO₂ feed gas, thereby contributing to improved N/C ratio in the LP recovery section.

It is also possible to supply a part of the CO₂ feed (e.g. 1 to 5%) to the LP section to decrease the N/C ratio.

Preferably, the gas from the gas/liquid contacting unit or units is supplied to the MP carbamate condensation section.

The construction of the stripper, reaction zone and condensation zone are as follows, which features, details and preferences apply to the existing plant, the modified plant, and the inventive plant.

The stripper is a shell-and-tube heat exchanger, with a falling film of urea solution in the tubes in operation, and with a liquid inlet at the top, a gas outlet at the top, a liquid outlet at the bottom, and a strip gas inlet at the bottom. The stripper contains an upper and a lower tube sheet for the tube bundle, with heating fluid in the shell side compartment between the tube sheets.

The strip gas used in the stripper is gaseous CO₂ feed in the existing plant, from a HP CO₂ compressor, that is in in the tubes in counter-current with the falling film of urea solution while the urea solution is heated. The stripper receives heating fluid, usually steam, on the shell side in operation. The steam is e.g. extracted from the HP CO₂ compressor.

Preferably at least 80% or at least 90% of the CO₂ feed is introduced directly in the stripper.

The reaction zone is provided by a urea reactor, or a part of a combined vessel also providing the condensation zone (e.g. a pool reactor), or a combination thereof.

The urea reactor, if used, is preferably a vertical urea reactor with one or more inlets at the bottom, a liquid outlet (e.g. provided by a downpipe) for withdrawing urea solution from the top, and with trays, and typically with a gas outlet at the top. The liquid outlet is connected to the liquid inlet of the stripper.

Typically, in operation, the liquid level in the urea reactor is at a higher elevation than the liquid inlet of the stripper, and this enables flow of liquid from the reaction zone to the stripper by gravity. The liquid level in the pool reactor is also at a higher elevation that the stripper inlet, to enable flow of liquid from the reaction zone to the stripper by gravity.

The synthesis section comprises a condensation zone, which is a separate HP carbamate condenser or a part of a combined vessel also providing a reaction zone (e.g. a pool reactor); both hereinafter also referred to as condenser. The HP carbamate condenser is a shell-and-tube heat exchanger with at least a first tube bundle, preferably with a first and a second tube bundle, condensation of gas from the stripper in the shell (at HP) and with MP urea solution in the tubes of the first tube bundle. The MP urea solution acts as cooling fluid for the condensation in the shell side space.

The HP carbamate condenser preferably comprises a first sparger for distributing gas from the stripper in the shell side space, for distributing the gas horizontally and below the tube bundle. The HP carbamate condenser preferably comprises an inlet for NH₃ feed in the shell side space, preferably with a second sparger for distributing the NH₃ horizontally, preferably arranged below the tube bundles. The shell side space in operation also receives MP carbamate solution from an MP carbamate condenser.

The first and second tube bundle are preferably U-shaped tube bundles.

The ends of the tube bundle may be attached to a tube sheet. Various configurations can be used for the tube-to-tubesheet connections, for example using sleeves, such as described in US 2015/0086440. In a further embodiment, the tubes are attached to a redistribution chamber as described in US 2020/0306663. Other configurations are also possible.

The first tube bundle receives MP urea solution in operation and the typically used second tube bundle is used for raising (LP) steam in operation. The first and second tube bundle are e.g. looped/nested, e.g. as described in WO 2023/219506. Preferably, the MP urea solution is supplied to the upper legs of the U-shaped first tube bundle, wherein the first tube bundle is e.g. the outer tube bundle.

In an embodiment, the plant comprises a separate reactor, and the condenser comprises a liquid outlet from the shell side space connected to an inlet of the reactor, and a gas outlet from the shell side space; optionally a part of the urea is already formed in a reaction zone in the condenser. In an embodiment wherein the condensation zone and reaction zone are combined in a single vessel, the vessel has a liquid outlet from the shell side space connected to an inlet of the stripper, and a gas outlet from the shell side space. An example of the combined vessel is a pool reactor, e.g. as described in U.S. Pat. No. 5,767,313. Preferably, the existing plant comprises a pool reactor. A pool reactor comprises for instance vertical partition plates (with apertures) which divide the reactor horizontally in compartments.

Typically, the U-shaped tube bundles extend through some but not all compartments of the pool reactor; i.e. the pool reactor comprises compartments between the bend of the U-shaped tube bundles and the shell. The compartments with the tube bundles provide the condensation zone, the compartments without the tube bundles provide for a reaction zone. The first sparger for gas from the stripper preferably extends through both the condensation zone and the reaction zone, e.g. through all compartments except the last compartment (the compartment that is most downstream for liquid). Thereby very elegantly, the gas from the stripper can provide heat for the endothermic urea formation reaction in the reactor part of the pool reactor.

The second sparger for instance extends through the condensation zone compartments. The partition plate of the last compartment may act as an overflow weir, with a liquid outlet at the bottom of the last compartment. The pool reactor typically has a gas outlet at the top. In operation, a liquid level is maintained in the shell side space, usually at the height of the top of the vertical partition plates. A gas discharge area is defined by the top of the partitions and the shell.

The MP treatment section comprises the gas/liquid separation unit and the gas/liquid contacting unit. The separation unit is e.g. a vessel with a gas outlet at the top and a liquid outlet at the bottom. The MP gas/liquid contacting unit comprises, e.g., a liquid inlet at an upper part, a packed bed below the liquid inlet, a gas inlet and liquid outlet below the packed bed, and a gas outlet at the top. The gas/liquid contacting unit is preferably an adiabatic unit.

The MP carbamate condensation section comprises preferably a shell-and-tube heat exchanger with gas to be condensed in the shell side space as a first MP carbamate condenser and urea solution to be heated in the tubes, such that the tube provides an evaporation stage. Preferably, the plant comprises a liquid flow line for urea solution directly or indirectly from the LP dissociator to said tubes (evaporation stage).

Preferably, the first MP carbamate condenser has an outlet for fluid connected to an MP carbamate separator. The liquid from the carbamate separator, i.e. MP carbamate solution, is supplied directly or indirectly to the shell side space of the horizontal submerged condenser, and the gas is supplied directly or indirectly e.g. to an absorber.

Preferably, the LP dissociator has a gas outlet connected to an LP carbamate condenser, and the LP carbamate condenser has an outlet with a liquid flow connection for LP carbamate solution to said absorber. Preferably, the absorber has a liquid outlet for LP carbamate connected to said first MP carbamate condenser.

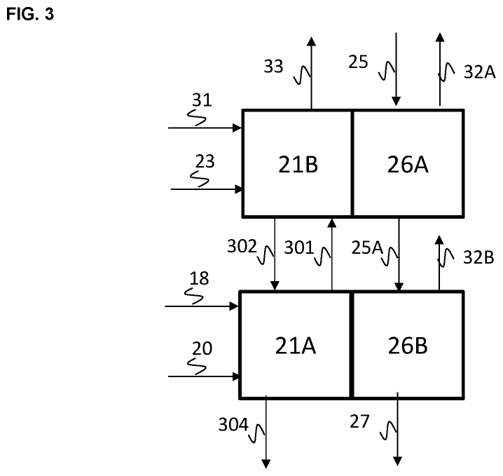

In a preferred embodiment (schematically shown in FIG. 3) the MP condensation section of the inventive process and inventive/revamped plant comprises a first MP carbamate condenser (21A) and a second MP carbamate condenser (21B) connected to receive non-condensed gas (301) from the first MP carbamate condenser. This embodiment can be used also in urea production plants and processes wherein the MP treatment section (9) receives stripped urea solution from the HP stripper. Hence, generally, in connection with this MP condensation section, the MP treatment section receives urea solution from the HP stripper and/or the reaction zone. Accordingly, for the MP condensation section of FIG. 3, the second liquid flow line is optional. For instance, the condensation section of FIG. 3 is used in the plant of FIG. 1, but with the second liquid flow line (8B) omitted.

Otherwise, all preferences for the plant of FIG. 1 apply also to the plant comprising the condensation section of FIG. 3. Hence, the MP carbamate condensation section of FIG. 3 is preferably used in the first aspect of the invention and is used in the second aspect of the invention, as discussed hereinbefore.

MP carbamate (302) from the second carbamate condenser (21B) is supplied to the first MP carbamate condenser (21A). The second carbamate condenser (21B) is operated at a lower pressure than the first MP carbamate condenser (21A), e.g. 1 to 5 bar lower. The second carbamate condenser (21B) is preferably in indirect heat exchanging contact with a first evaporation stage (26A) that receives urea solution (25), typically from the LP recovery section, and typically at a pressure below 1.0 bara. The second carbamate condenser (21B) and the first evaporation stage (26A) are e.g. provided as a shell-and-tube heat exchanger. The first evaporation stage (26A) has an outlet for water vapor (32A) that is sent e.g. to an absorber. The heated urea solution (25A) from the first evaporation stage (26A) is further heated in a second evaporation stage (26B) in indirect heat exchanging contact with the first MP carbamate condenser (21A). The second evaporation stage (26B) and the first MP carbamate condenser (21A) are e.g. provided as a shell-and-tube heat exchanger. In the revamp, preferably this heat exchanger is added. The second evaporation stage (26B) has outlet for vapor (32B) and a separate outlet for a concentrated urea solution (27), that is supplied e.g. to a further heating stage that uses LP steam (such as raised in the second tube bundle) or MP steam for further water evaporation. Non-condensed gas (33) from the second carbamate condenser (21B) is supplied for instance to an absorber. MP carbamate solution (304) from the first MP carbamate condenser (21A) is supplied to the HP synthesis section, in particular to the condensation zone.

The second carbamate condenser (21B) receives an aqueous solution in operation, in particular carbamate solution (31) from the LP recovery section, and preferably from the LP processing of both the stripped and non-stripped urea solution. The water contained in the carbamate solution prevents carbamate precipitation in the MP carbamate condensation section.

The off-gas (23) from the synthesis section, preferably from the condensation zone, is supplied, preferably exclusively, to the second carbamate condenser (21B). This off-gas (23) contains inert gas, such as O₂ or air used for passivation in the synthesis section.

The first MP carbamate condenser (21A) receives the gas (18) from the gas/liquid separation unit (11) and the gas (20) from the gas/liquid contacting unit (20). The MP carbamate condenser (21A) accordingly preferably receives substantially no inert gas. This contributes to a higher condensation temperature and higher urea concentration that can be achieved in the second evaporation stage (26B).

It was found that very elegantly, urea solution can be increased, as an example, from 72 wt. % urea to a concentration of at least 78 wt. %, e.g. to about 80 wt. % urea with only process-process heat integration in this embodiment (wt. % urea including biuret, from the inlet of the first evaporation stage (26A) to the outlet of the second evaporation stage (26B), and for a HP stripper bypass fraction in the range of about 35% i.e. such amounts of total urea solution bypassing the HP CO₂ stripper. This advantage is obtained independent of supplying urea solution from the reaction zone or from the HP stripper to the MP treatment section.

The revamp method may involve modifying the existing MP condensation section to obtain an MP condensation section as mentioned. In particular, in an embodiment, the existing MP condensation section (21) comprises a first MP carbamate condenser (21A) and the method comprises adding a second MP carbamate condenser (21B) connected to receive non-condensed gas (301) from the first MP carbamate condenser, with a connection for MP carbamate (302) from the second carbamate condenser (21B) to the first MP carbamate condenser (21A). In this embodiment, in the modified plant, the second carbamate condenser (21B) is in indirect heat exchanging contact with a first evaporation stage (26A) that receives urea solution (25), and the first evaporation stage (26A) has an outlet heated urea solution (25A) connected to a second evaporation stage (26B) that is in indirect heat exchanging contact with the first MP carbamate condenser (21A). The modified plant comprises a gas flow line for off-gas (23) from the synthesis section, preferably from the condensation zone, to the second carbamate condenser (21B), and a gas flow line for gas (18) from the gas/liquid separation unit (11) and gas (20) from the gas/liquid contacting unit (20) directly or indirectly to the first MP carbamate condenser (21A). Note that the inventive plant can also be constructed in other manners. For instance, when modifying an existing ULE plant, the existing MP carbamate condenser could also be used as the first MP carbamate condenser.

As used herein, for process streams (i.e. not for steam lines), high pressure (HP) is above 100 bar, for instance 120 to 300 bar, typically 150 to 200 bar. Medium pressure (MP) is for example 10 to 70 bar (including intermediate pressure of 30 to 70 bar), in particular 15 to 30 bar, and low pressure (LP) is for example 0 to 10 bar, in particular 1 to 8 bar or 2 to 5 bar. All pressures are bar absolute (bara).

As used herein, the stripping efficiency alpha=(2*wt. % urea/60)/((2*wt. % urea/60)+(wt. % NH₃/17)), measured at the liquid outlet of the stripper, wherein wt. % NH₃ includes all ammonia species including ammonium carbamate. Biuret is lumped with urea because the amounts of biuret are very low. The term ‘stripping efficacy’ could also be used. A higher stripping efficiency may be achieved with higher steam consumption.

The N/C ratio (NH₃: CO₂ ratio) of the reaction zone reflects the composition of the so-called initial mixture before urea production, consisting only of NH₃, CO₂ and H₂O, as used in the art of urea plants, and is the molar ratio, and is measured at the outlet of the reaction zone for urea synthesis solution.

The free N/C ratio of urea solutions is the molar ratio NH₃: CO₂, on the basis of N and C in the solution including ammonium carbamate but excluding urea and biuret. Hence, the free N/C ratio refers to the molar ratio NH₃: CO₂, on the basis of N and C, based on CO₂, NH₃ and carbamate in the solution; and can also be referred to as the N/C atom ratio based on CO₂, NH₃ and carbamate in the solution.

The N/C ratio for gas streams is the molar ratio of NH₃ to CO₂.

The N/C ratio for carbamate condensers, such as the first condensation compartment, is the molar ratio NH₃ to CO₂ including as carbamate at the outlet for carbamate liquid.

Carbamate, as used herein, refers to ammonium carbamate, as that term is used in the art.

Condensation in a carbamate condenser refers to so-called carbamate condensation, which involves the reaction of NH₃ and CO₂ into carbamate forming carbamate solution. Carbamate decomposition refers to the dissociation reaction of carbamate into NH₃ and CO₂.

The terms ‘typically’ and ‘preferably’ and derived forms indicate non-mandatory features. The term ‘or’ generally is not exclusive, i.e. permits the combination of both elements.

Preferably the urea production process is carried out in the inventive urea plant. All preferences for the urea production process apply equally for the urea plant. All preferences and details indicted for equipment parts in connection with the urea production process, apply equally for the urea plant.

As used herein, the term ‘in series’ permits the presence of additional units, also between the recited units, and permits that one or more recited units are combined in a single apparatus (typically have separate zones corresponding to each recited unit). Moreover, the urea solution in operation typically flows through the series of units in the recited order.

### EXAMPLES

The invention will now be further illustrated by the following non-limiting example(s). These examples do not limit the invention and do not limit the claims.

### Example 1

Based on model calculations, the following steam consumption results can be achieved.

Case 1—reference: an existing ULE plant with a pool reactor is operated at 110% plant capacity (i.e. 110% of design capacity) with a stripper steam consumption of 630 kg steam/ton urea (~25 barg saturated steam from battery limit; stripper shell side pressure is about 14 barg), and a stripping efficiency ~63% at stripper. The design of the plant was according to FIG. 1 without the second liquid flow line (8B).

Case 2—inventive: the plant is modified according to the invention (by adding the second flow line bypassing the HP stripper) to achieve 135% plant capacity without additional reaction volume. The HP CO₂ stripper was operated with stripping efficiency ~70%.

The steam consumption was 670 kg/ton (~25 barg saturated steam from battery limit; stripper shell side pressure is about 15 barg). About 35% of the urea solution from the reactor is sent directly to the MP section, bypassing the HP stripper. The design of the plant was according to FIG. 1 with the added the flow line (8B).

Case 3—inventive: the plant is modified according to the invention (first aspect) to achieve 150% plant capacity with additional reaction volume, stripping efficiency ~78% at stripper). The stripper steam consumption is 680 kg/ton (~25 barg saturated steam from battery limit; shell side pressure is about 17.5 barg).

For this case due to the lower LP steam pressure, extra LP steam (~3.5 barg) from battery limit of about 100 kg/ton is imported. About 40% of urea solution (unstripped) is sent directly to the MP section (MP flashing followed by the first tube bundle), bypassing the HP stripper through the added second flow line. The design was according to FIG. 2; with a vertical urea reactor added in the second liquid flow line (8B) upstream of the valve (14A) to provide the additional reaction volume.

1-15. (canceled)

16. A urea production plant comprising:
a high pressure (HP) synthesis section comprising a reaction zone, a condensation zone, and a HP CO₂ stripper, wherein the condensation zone is provided by a shell-and-tube heat exchanger comprising a shell side space and a first tube bundle;
a gas flow line for gas from the HP CO₂ stripper to said shell side space;
a first liquid flow line for a first part or all of the urea solution from the reaction zone to said HP CO₂ stripper;
an MP treatment section, wherein the MP treatment section is connected to receive urea solution from the HP synthesis section, wherein the MP treatment section comprises, in series for urea solution, a medium pressure (MP) flashing section, the first tube bundle, an MP gas/liquid separation unit, and an MP gas/liquid contacting unit, and
an MP carbamate condensation section connected to receive gas from the MP gas/liquid separation unit and the MP gas/liquid contacting unit,
wherein the MP condensation section comprises:
a first MP carbamate condenser;
and a second MP carbamate condenser connected to receive non-condensed gas from the first MP carbamate condenser, with a connection for MP carbamate from the second carbamate condenser to the first MP carbamate condenser, wherein the second carbamate condenser is in indirect heat exchanging contact with a first evaporation stage that receives urea solution, wherein the first evaporation stage has an outlet for heated urea solution connected to a second evaporation stage that is in indirect heat exchanging contact with the first MP carbamate condenser and wherein the plant comprises:
a gas flow line for off-gas from the synthesis section to the second carbamate condenser, and
a gas flow line for gas from the gas/liquid separation unit and gas from the gas/liquid contacting unit directly or indirectly to the first MP carbamate condenser.

17. A urea production process carried out in a plant according to claim 16, comprising:
reacting NH₃ and CO₂ to form urea in said reaction zone and condensation zone;
supplying all or a part of the CO₂ feed to the HP CO₂ stripper; and
supplying urea solution from the synthesis section to the MP flashing section.

18. The urea production process of claim 17, wherein the urea solution is supplied from the reaction zone and/or from the HP stripper to the MP flashing section.

19. The urea production process of claim 17, wherein the second MP carbamate condenser (21B) is operated at a lower pressure than the first MP carbamate condenser (21A).

20. The urea production process of claim 19, wherein the second MP carbamate condenser (21B) is operated at a pressure 1 to 5 bar lower than the first MP carbamate condenser (21A) 1 to 5 bar lower.

21. A urea production plant according to claim 16, wherein the MP treatment section (9) is connected to receive urea solution from the reaction zone and/or the HP CO₂ stripper.

22. A urea production plant according to claim 16, wherein the gas flow line for off-gas (23) extends from the condensation zone to the second carbamate condenser (21B).
